(19) United States
(12) Patent Application Publication
(10) Pub. No.: US 2018/0214654 A1
(43) Pub. Date: Aug. 2, 2018

(54) NICU CPAP Cannula 360 Circuitry Support System

(71) Applicants: Robert Dean Talbot, (US); Kirk Gustafson, (US)

(72) Inventors: Robert Dean Talbot, Wasilla, AK (US); Kirk Gustafson, Fairbanks, AK (US)

(21) Appl. No.: 15/418,665

(22) Filed: Jan. 27, 2017

(51) Int. Cl. A61M 16/06; A61M 16/08; A61F 9/04

(52) U.S. Cl. CPC A61M 16/0683; A61M 16/0666; A61M 16/0875; A61M 2209/08; A61M 16/0694; A61M 2240/00; A61F 9/045

(57) ABSTRACT

An improvement in the apparatus to secure a nasal cannula used with continuous positive airway pressure machines used with infants in neonatal intensive care units. A soft knit hat with slots is used to secure air hoses feeding the cannula. Elastic straps thread through the slots to secure the hoses to the infant's head. A chin strap is used to hold the baby's mouth closed to prevent air leakage while the cannula is in use. The hat also includes a set of horizontal slots in the center of the hat to attach a required eye shield. The device allows the infant to sleep on one side without hoses and tubes getting in the way. It also allows a parent to hold the child comfortably. Hoses can be secured to a chair or bed with a hose strap.

### CROSS REFERENCE TO RELATED APPLICATIONS

Not Applicable

### STATEMENT REGARDING FEDERALLY SPONSORED RESEARCH AND DEVELOPMENT

Not Applicable

### BACKGROUND OF THE INVENTION

### 1. Field of the Invention

The present invention relates to a cannula circuitry support system for use with continuous positive airway pressure (CPAP) machines used with infants in neonatal intensive care units (NICU) to secure the apparatus to an infant's head and more particularly to cannula circuitry support systems including a nasal cannula, hat, straps, chin strap and an eye shield.

### 2. The Description of the Prior Art

At this time, children in a NICU face many challenges. One challenge includes the functionality of devices currently in use and the lack of others, which could be helpful. The major components currently in use, including: an eye shield, CPAP circuit straps, a CPAP circuit stabilizer, and a CPAP nose cannula.

CPAP continuous positive airway pressure is a device sometimes ran through a bubbler to help the aid in breathing of infants due to immature or damaged lung tissue. The CPAP circuit (hoses) from the CPAP machine delivers a positive pressure via one hose through a cannula in the nose and into the lungs, and a return hose on the other side of the cannula which returns air back to the machine, which monitors air leakage, among other vital information.

The CPAP cap in use today is worn so that the CPAP circuit from the CPAP machine can be supported close enough to the cannula to secure placement and help support the cannula from falling out or moving causing leakage and thus not being able to deliver the correct amount of oxygen to lungs. Issues with the CPAP cap in use today are the lack of expandability from micro-premature children to full term child growth. With respect to the wide range of child cranial size and development, the CPAP cap should be able to be adjustable to provide a secure adjustable fit with the ability to secure the CPAP circuit without endangering the child or the nurses. Many NICU hospitals use the safety pins with rubber band to secure the CPAP circuit to the hat, which poses safety issues for nurses and patient's. Another issue with the CPAP cap is ensuring it is secured tightly enough to the head so that it does not move, thus causing a bigger leakage and or cannula displacement.

The chin strap currently in use have many problems, including size, shape, placement, and the method of securing. All of these components play a part in how efficiently the children receive oxygen. For example, retinopathy of prematurity (ROP) is the condition that plagues the eyes of some premature children and the oxygen supplied by the CPAP machines plays a vital role in development of the vessels in the eyes. Too much oxygen can cause the vessels to grow incorrectly, tearing the retina and causing poor eyesight and even blindness. The first issue with the chip strap is the adjustability of the strap-not only for micro-premi's but full gestational children that need the care of the NICU. The second is the ability of the chinstrap remain secured to the child, which can lead to “D STSAT” (the loss of oxygen sufficient for the child, which causes the heart to slow putting the child at risk of injury or death). The third issue is the problem of chin straps that slide back toward the esophagus during movement, which can choke the child and causes issues with the esophagus. Another issue with the chin strap is that it can be overtightened.

Eye shields are used to protect the baby's eyes while undergoing light therapy. Often, a foam eye glasses or just a piece of cloth is used. However, this can create issues of pressure in the eyes from the tightening of these items. Moreover there is a problem in fitting these items to smaller micro-premi babies. Another problem with the cloth and foam glasses is the possibility of their falling off while the child is moving. An eye shield is important to those children that need their eyes protected while receiving photo therapy for jaundice or waiting for the optometrist to “clear” the baby's eyes from ROP if additional protection to the eyes is needed.

Also, as noted above, attaching the CPAP circuit onto the hat has had problems. Many times safety pins or rubber bands are used to hold the CPAP circuit in place. Use of pins are problematic because of safety and infection issues.

The circuit stabilizer currently used to secure the CPAP circuit to a chair in conjunction with a mother holding her child (e. g., kangaroo care) has issues. Although the experience and data shows a greater child development when parents are able to hold their children (creating a family bond), the hoses that are not secured creates leakage and sometimes dislodges the cannula from the child's nose, discontinuing the oxygen to the child. This causes the child to “dstat” (the loss of oxygen in the bloodstream). Holding your child in this situation is very stressful for the parents and staff, because every time the child “dstats”, nurses or respiratory therapist rush in to fix and administer the oxygen back to the child. Stress and anxiety can be overbearing to the point it can make a parent reluctant to hold his or her child for fear they would cause further issues.

### BRIEF DESCRIPTION OF THE INVENTION

The instant invention overcomes the above difficulties. It is an improvement in the handling and application of the NICU nasal cannula and securement apparatus for the infant patient. The invention can be utilized in many configurations to suit the needs of the patient and hospital staff.

The invention includes a CPAP hat, a chin strap, an eye shield, CPAP circuit straps, a CPAP circuit stabilizer, and a CPAP nose cannula. Unlike those devices used in the prior art, however, the instant invention makes significant improvements.

The instant invention uses a soft, knit, cotton hat that has two sets of slots in the center to allow for attachment of an eye shield. In addition, three slots on each side of the hat allow for a single hose attachment or a double hose attachment to the hat using hook and loop type fasteners. The hat has a larger slot in the top that allows a doctor to preform a palpable fontanelle check without having to unstrap the hoses and remove the hat (a fontanelle check is performed by inserting a finger in the slit in the cap and feeling the top of the patient's head). As noted above, the hat has two rows of slots; by rolling the hat, it can be adjusted for smaller heads.

A chin strap is comprised of a soft, double-sided, foam-lined cotton fabric material with a slit in each side, to allow the strap of a crown strap to be secured. The center of the chin strap also has a small hook and loop type fastener button, which attaches to an opposing hook and loop type fastener button between two layers of DUODERM, which is a hydrocolloid dressing manufactured by ConvaTec of Deeside, UK. The double layered DUODERM adheres to the patient's chin. The strap is then secured to the patient with opposing hook and loop type fastener buttons. The crown strap is made from a soft, stretchable fabric material. The center of the crown strap has 3 long slits that allow for air movement.

The crown strap sits towards the back of the head and loops through the chin strap via a hook and loop type fastener strips.

An eye shade is comprised of a soft, double-layered dark cotton material that shields the infant's eyes from light when needed. The shade has two long tapered strips that slide into the horizontal center slots in the cap, thus allowing a health care professional to insert the tab into the slots on the cap. These strips have a thin piece of plastic is sewn between the pieces of fabric for rigidity. The eye shade can be slid up or down for proper placement on the patient's face.

The CPAP circuit strap secures the CPAP hoses to the bed or chair while the child is being held. The strap is comprised of three thin layers of plastic. The plastic has slits in each end that allow the strap to be secured to a bed or chair via long thin hook and loop type fastener straps. The strap has a long slit in the center to allow two hose clamps to move in any direction as needed. Hose “C” clamps snap onto the hoses for a secure fit. On the bottom of the clamp is a circular piece of plastic that swivels and slides in the center plastic groove. When sandwiched together the clamp is tight enough so that it does not swivel and move without some effort.

Finally, a nasal cannula is provided that is single-sided and can be easily rotated in any direction over 360 degrees. The single-sided cannula allows the patient to lay on his or her side as well as allowing parents to hold the child in a natural position without hoses getting in the way. It also allows for important skin to skin contact. The side of the cannula with the tubes is secured to the soft cotton knit hat via the vertical of slots in the hat, and cotton, elastic and hook and loop type fastener strap. The opposing side can be secured with a strap made from DUODERM, adhered the patient's cheek.

### BRIEF DESCRIPTION OF THE DRAWINGS

FIG. 1 is a front perspective view of the device on an infant patient.

FIG. 1a is a front perspective view of the device on an infant patient showing the placement of the eye shield on the infant.

FIG. 2 is a right side view of a patient's head showing the placement of the device.

FIG. 2a is a right side view of a patient's head showing the placement of the device including the eye shield.

FIG. 3 is a left side view of a patient's head showing the placement of the device including a DUODERM strap patch.

FIG. 3a is a left side view of a patient's head showing the placement of the device including the eye shield.

FIG. 4 is a detail view of the soft knit cap in an unrolled position.

FIG. 4a is a detail view of the soft knit cap shown rolled once.

FIG. 4b is a detail view of the soft knit cap shown rolled twice.

FIG. 5 is a top view of a short strap that secure a single hose to the hat through the slots therein.

FIG. 5a is a side view of the short strap.

FIG. 5b is a top view of a long strap that secure a two hoses to the hat through the slots therein.

FIG. 5c is a side view of the long strap.

FIG. 5d is a detail view of one of the straps shown wrapped around a hose and secured thereon.

FIG. 6 is a front view of the eye shield.

FIG. 7 is a top view of the crown part of the chin strap.

FIG. 8 is a detail view of the lower part of the chin strap.

FIG. 9 is a detail view of the DUODERM chin patch.

FIG. 10 is a top view of the cannula used with the invention.

FIG. 11 is a side view of the cannula used with the invention.

FIG. 12 is a front view of the cannula used with the invention.

FIG. 13 is a side perspective view of the cannula used with the invention.

FIG. 14 is a bottom perspective view of the cannula used with the invention.

FIG. 15 is a perspective view of the cannula used with the invention showing the tube separated from the prongs.

FIG. 16 is a perspective view of the device with the stabilizing strap attached.

FIG. 17 is a top view of the stabilizing strap.

FIG. 17a is a side view of the stabilizing strap.

FIG. 18 is a top detail view of the stabilizing strap showing an alliterative attachment structure.

FIG. 19 is a top view of the top plate used with the stabilizing strap.

FIG. 20 is a detail view of the center plate used with the stabilizing strap.

FIG. 21 is a detail view of the bottom plate used with the stabilizing strap.

FIG. 22 is a detail view of a “C” clamp used with the stabilizing strap.

### DETAILED DESCRIPTION OF THE INVENTION

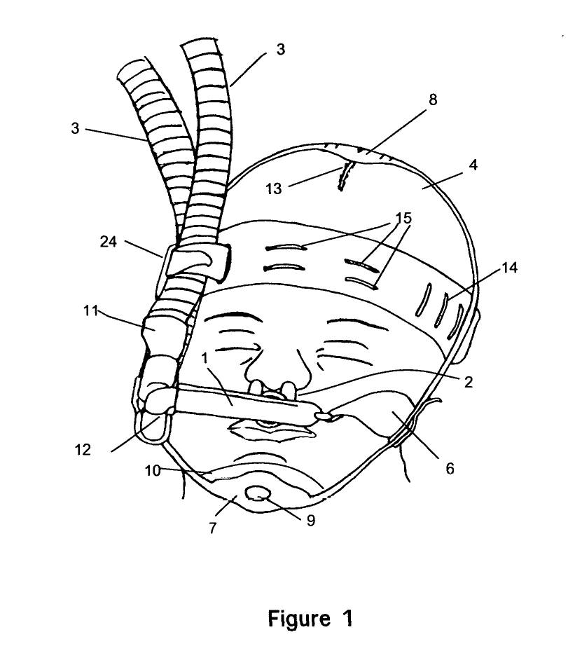

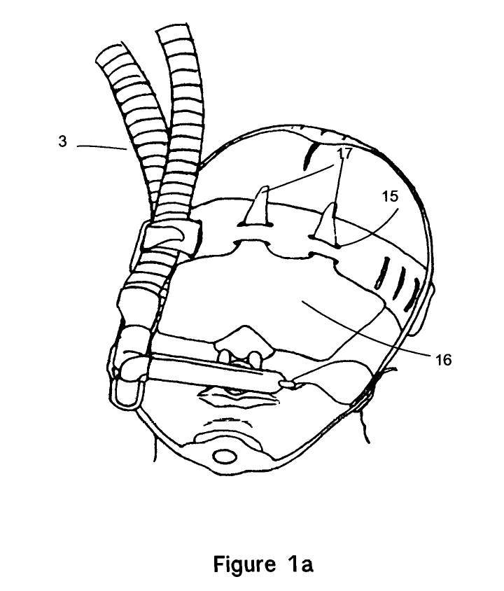

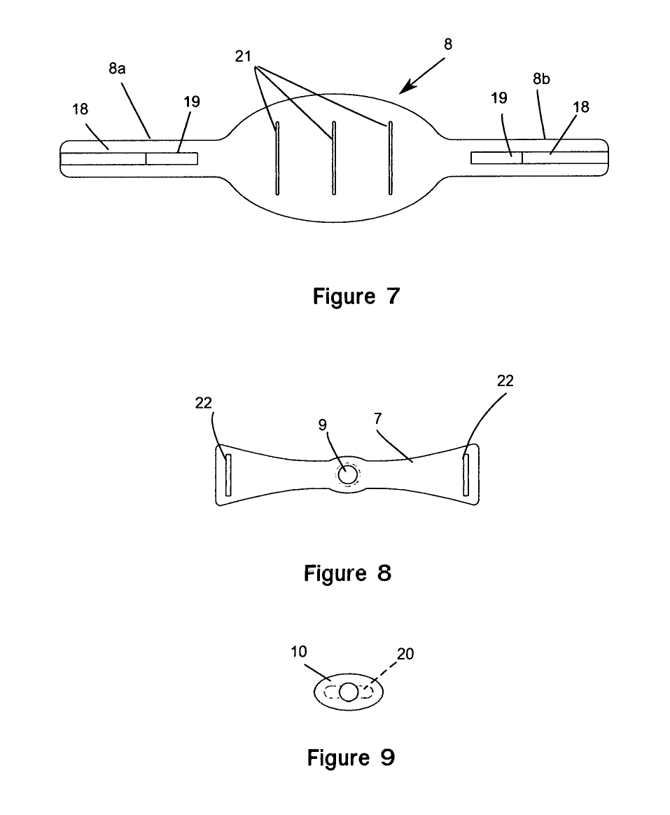

Referring now to the drawings, FIG. 1 is the front perspective view off the invention and the circuitry support system invention on the infant patient. This view shows the insertion of the nasal prongs 2 of the invention into the nasal cavity of the infant and the attachment of two circuitry hoses 3 secured to the cap 4 with elastic and hook and loop type fastener straps 27 or 30. The opposing side of the cannula 1 is secured to the patient's cheek with a DUODERM strap 6. This view also shows the chin strap 7 attached to a crown strap 8, using hook and loop type fastener straps 18 and 19 see, FIG. 7. In this view a small hook and loop type fastener button 9 is shown in the center of the chin strap. The hook and loop type fastener button 9 secures to a DUODERM chin patch 20 with an opposing hook and loop type fastener button 1. see FIG. 9. Hoses 3 are connected to a hose adapters 11 and elbows 12.

A long slot 13 in the top of the cap 4 allows a doctor to perform a palpable fontanelle check without having to remove the hat. Also in this view are the three vertical slots 14 on each side of the hat 4 that receive the hose straps 27 or 30, as discussed above, that secure the hoses 3 to the patient's head. The four horizontal slots 15 in the center of the hat 4 are for the insertion of the eye shield 16, as shown in FIG. 1a, for example.

FIG. 1a is a detail view of the invention with the placement of the eye shield 16. The eye shield has tabs 17 (see FIG. 6). The tabs 17 are inserted into the hat 4 through slots 15, which allow for vertical adjustments of the eye shield.

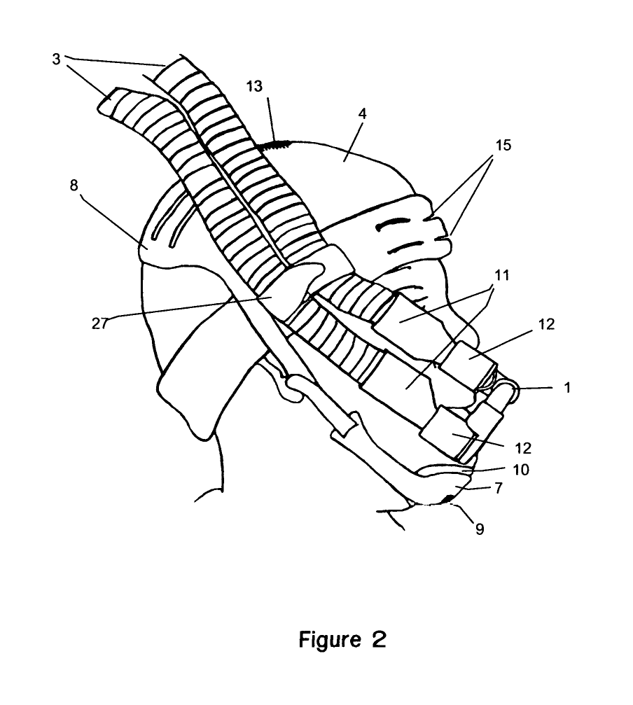

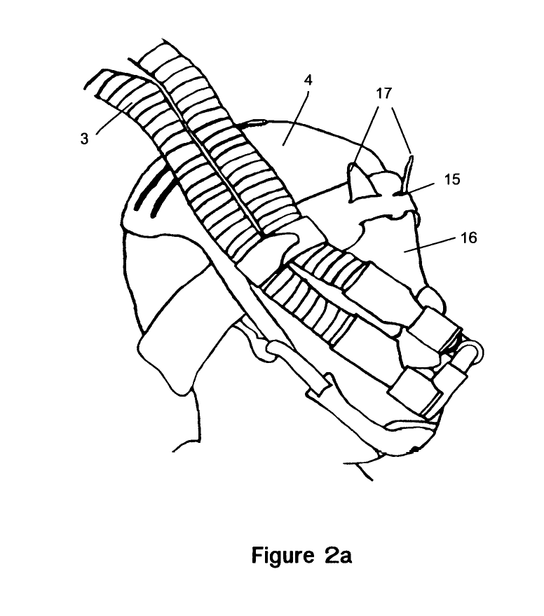

FIG. 2 is a right side view of the patient's head showing the placement of the invention. This view shows the double hose strap 30 being used to secure the hoses 3 to the cap 4 through slots 14.

FIG. 2a of the claim shows the same parts as FIG. 2 but with the placement of the eye shield 16 on the infant and the tabs 17 inserted into the slots 15 on the hat 4.

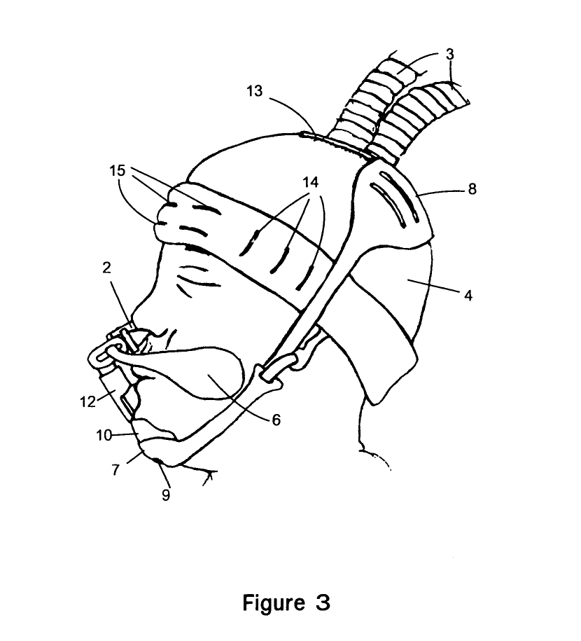

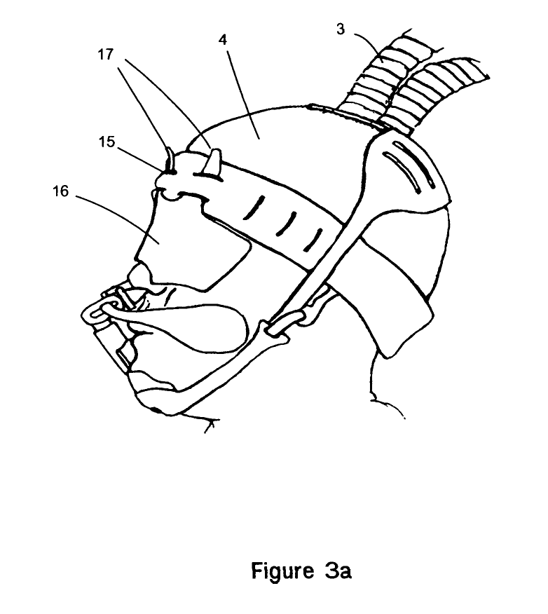

FIG. 3 is a left side view of the patient's head showing the placement of the invention. In this view you can see how the cannula 1 is secured to the patient's cheek with a DUODERM strap patch 6.

FIG. 3a is a left side view of the patient's head showing the placement of the invention with the eye shield 16 as discussed before.

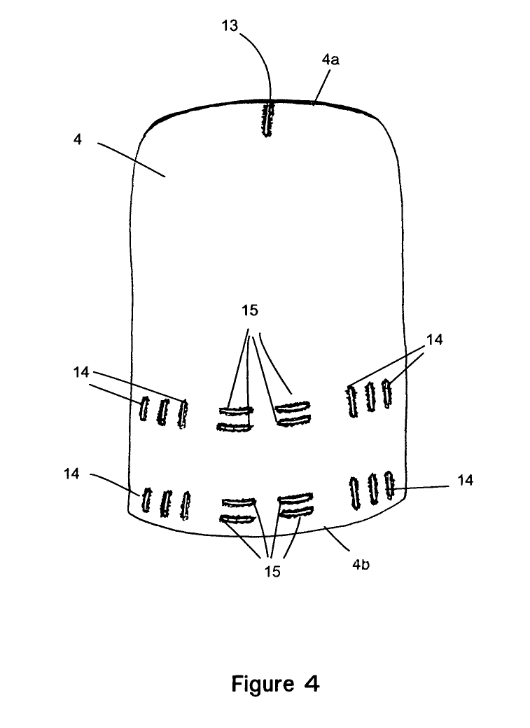

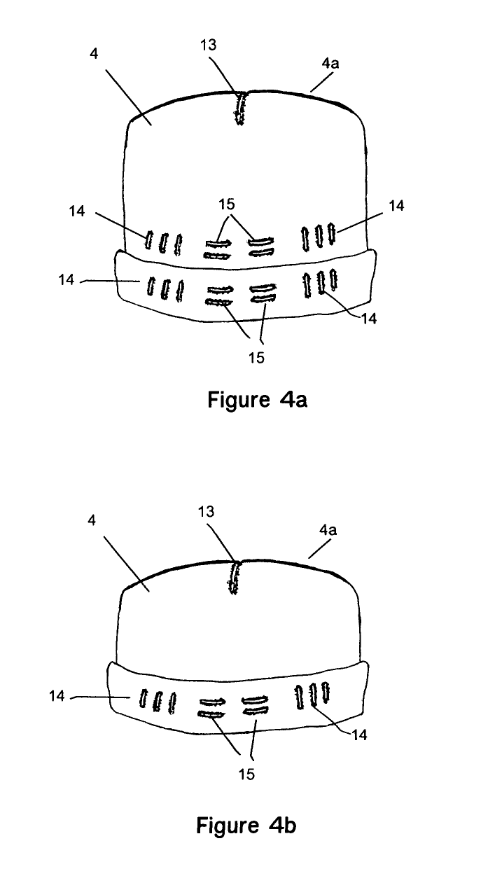

FIG. 4 shows the soft knit cap 4 in the unrolled position. The cap 4 has a top 4a and a bottom rim 4b. This illustration shows two rows of 10 slots. The vertical slots 14, (there are six on each side) are for the attachment of the circuit hose straps 27 or 30. The horizontal slots 15 are for the attachment of the eye shield 16. The eye shield is attached by inserting the tabs 17 into the horizontal slots 15. As noted above, on the top of the hat is a long slot 13, which allows a doctor to preform a palpable fontanelle check without having to unstrap the hoses and remove the hat. This figure shows the hat at its full extent; when worn by patient it is rolled up at least once, see FIGS. 4A and 4B.

FIG. 4a shows the hat rolled once and FIG. 4b shows the hat rolled up the maximum of two times. This allows the hat securely to fit a small infant's head. When rolled twice, as shown, and a new set of slots 15 are positioned to allow for the insertion of the eye shield 16 and the straps 27 or 30.

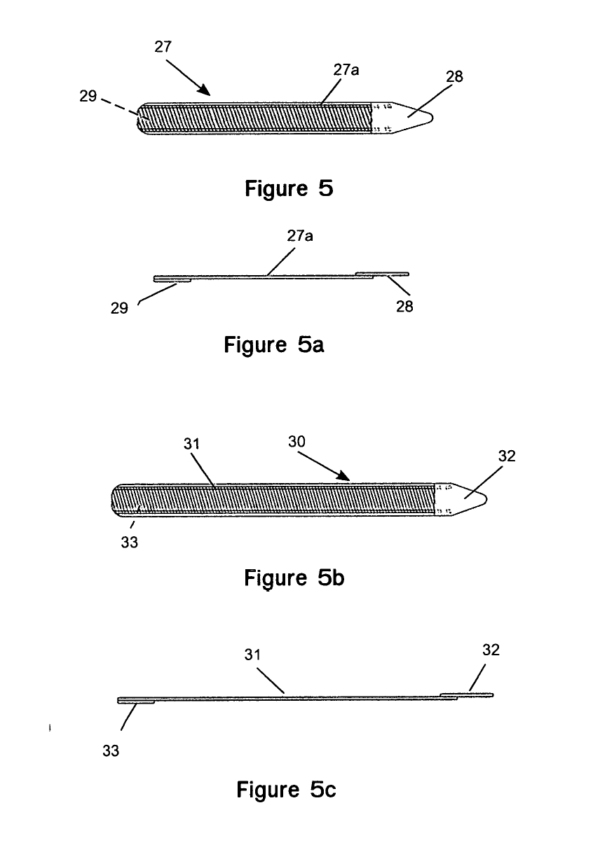

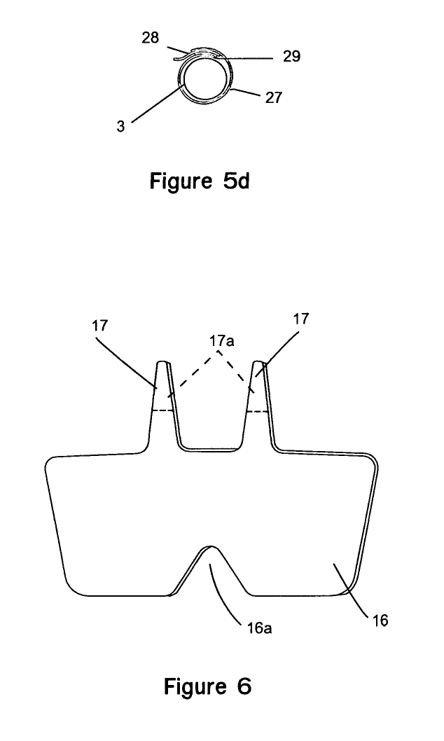

FIG. 5 is a top view of a short strap 27 that secures a single CPAP circuitry hose 3 to the hat 4. FIG. 5a is a side view of the short strap. Each strap is made up of a cloth elastic material 27a. The strap 27 has a pointed end that has a loop type piece of hook and loop type fastener 28 cut into a “V” shape and sewn to the fabric elastic as shown. Placing the loop portion of the hook and loop type fastener makes it easy to thread the strap through the slots 14 of the cap 4. The hook portion 29 of the hook and loop type fastener is sewn to the other side of the strap 27 as shown.

FIG. 5b is a top view of a long strap 30 that secures two CPAP circuitry hoses 3 to the hat 4. FIG. 5c is a side view of the long strap. Each strap is made up of a cloth elastic material 31. The strap 30 has a pointed end that has a loop type piece of hook and loop type fastener 32 cut into a “V” shape and sewn to the fabric elastic as shown. Placing the loop portion of the hook and loop type fastener makes it easy to thread the strap through the slots 14 of the cap 4. The hook portion 33 of the hook and loop type fastener is sewn to the other side of the strap 30 as shown.

FIG. 5d is a detail view the elastic straps 27 or 30 rolled up around the hoses and how the apposing hook and loop type fastener pieces 28 and 29 or 32 and 33 adhere to each other.

FIG. 6 is a front view of the eye shield 16. The eye shield 16 is made from double layered opaque soft material sewn together. The eye shield has a cutout 16a at the bottom allowing for a closer fit to the eyes of patient. The two long tabs 17 incorporate a thin piece of plastic 17a sewn between the layers at the tip so that the tab is more easily inserted into the slots 15 of the cap 4.

FIG. 7 is a front view of the top crown part of the chin strap 8. This piece is made from a soft flexible and stretchable material This chin strap has two long side straps 8a and 8b that have hook and loop type fastener strips 18 and 19. The hook and loop type fastener strip 18 is the loop type fastener and part 19 is the hook part of the hook and loop type fastener. Having loop type 18 towards the end allows for the strap to early be inserted into the slits 22 of the lower part of the chin strap 9 FIG. 8). Both hook and loop type fastener strips 18 and 19 are sewn to the front face of the crown strap, as shown. The crown part of the chin strap also has three long vertical slits 21 that allow for ventilation. They also allow the strap to conform to an infant's head when slightly stretched.

FIG. 8 is a front view of the lower part of the chin strap 7. The lower chin strap is made from soft double layered material with a this foam layer sandwiched between. The lower chin strap has 2 vertical slits 22 that allow for insertion of the hook and loop type fastener ends 18 and 19 on the crown strap piece 8. In the center of the strap 7 is a small round loop part of hook and loop type fastener 9 sewn between the layers of material. This button 9 attaches to the loop part of hook and loop type fastener 10 of the DUODERM patch 20 of FIG. 9.

FIG. 9 is a front view of the DUODERM chin patch 20 that adheres to the infant's chin. In the center of the double layered DUODERM patch is a butterfly shaped hook and loop type fastener 10, which is a loop part of hook and loop type fastener. The two parts of hook and loop type fastener 9 (FIG. 8, and 10 (FIG. 9) are secured together keep the chin strap in place on the infant.

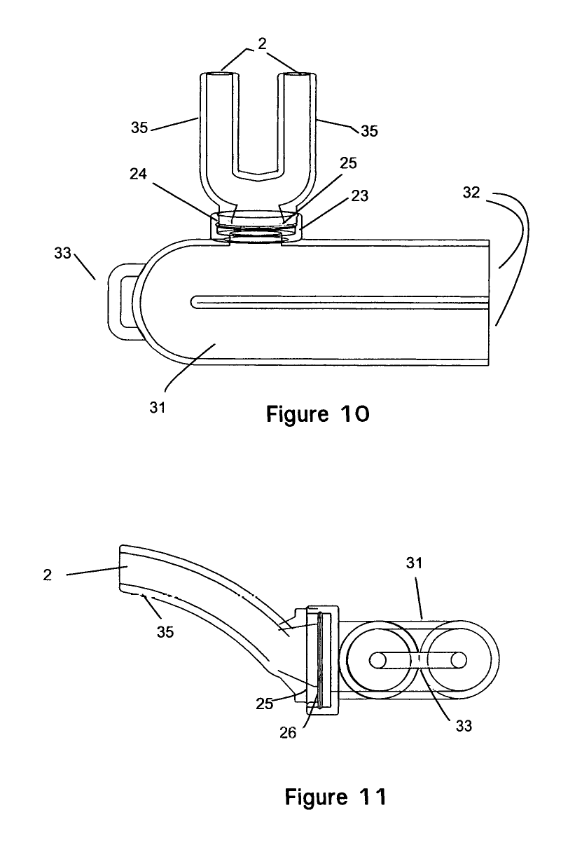

FIG. 10 is a top view of the cannula 1 used with the invention. This cannula is preferably made from a softer clear plastic and consists of two rotating pieces that are connected at threaded portions 23 and 26. Clear plastic allows for monitoring of the cannula for moisture or obstructions. The nasal prongs 2 are partially coated with soft hypoallergenic open cell polyurethane foam 35. The foam tips allow for expanding the width of the cannula prongs 2 to securely, but softly, expand into the nasal cavity for a snug fit to each individuals nostril. The nasal prongs 2 are made in three different sizes to fit range of infants from micro preemie to full term infants. The clear soft plastic cannula tube 1 has an inside diameter of ¼″ so that it fits the already available elbows 12 (see, e. g., FIG. 1). The ¼″ tubing has a 180 degree elbow 31 that allows for the intake tube and exhaust tubes 3 (see e. g., FIG. 1) to be positioned on the side of the patient's head.

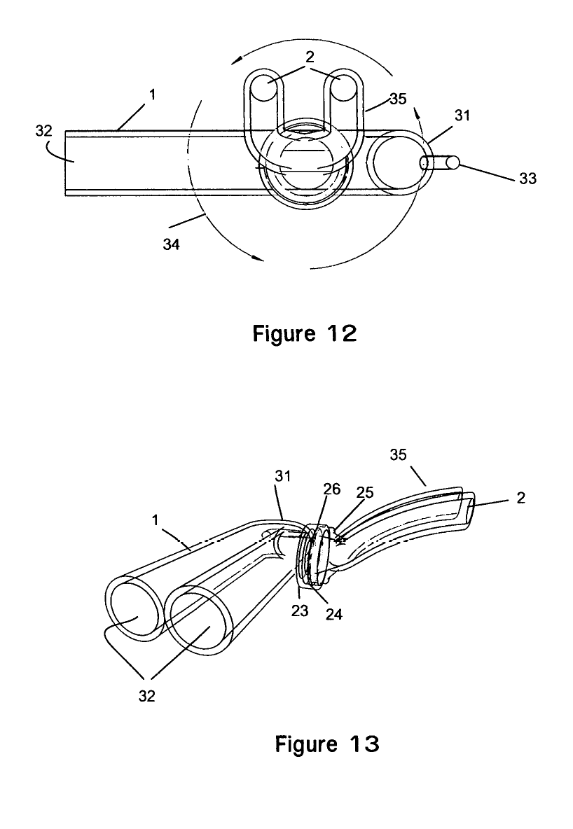

On the side of the tube 32 has circular fixed collar ring 23 with a “V” groove 24 cut into the inner surface. When attached to the nasal prong 2, the inner ring 25 and the “V” ridge 26 on the bottom of the nasal prongs 2 (see FIG. 15), allow the apparatus to rotate 360 degrees so that the cannula can be placed on either side of patient's face, see FIG. 12 with the arrows 34. The movement of the cannula can be done while the nasal prongs 2 are left in place on the patient. The raised “V” ridge 26 in the circular ring 25 on the base of the nasal prongs 2 securely snaps into the “V” groove 24 of the collar ring 23. FIG. 10 also shown a mounting strap loop 33 into which a strip of DUODERM 6 (see, e. g., FIG. 1) can be attached to secure the end of the cannula to the patient's cheek to stop the cannula from being dislodged.

FIG. 11 is a side view of the cannula used with the invention. In this view the loop 33, which allows a DUODERM strap 6 to be attached is shown extending from the end of the cannula. The collar ring 23, which snaps onto ring 25 of the nasal prongs 2 is also shown.

FIG. 12 is a front view of the cannula used with the invention. This view is a view that shows the device extending downward from the patient. As noted above, the cannula tube 1 is attached to the nasal prongs 2 via interlocking collar 23 and inner ring 25. Circular line 34 illustrates how the cannula tube 1 can be rotated with respect to the nasal prongs 2. As noted above, this allows the tubes 32 to be positioned on either side of the patient.

FIG. 13 is a side perspective view of the cannula used with the invention. It illustrates the components discussed above.

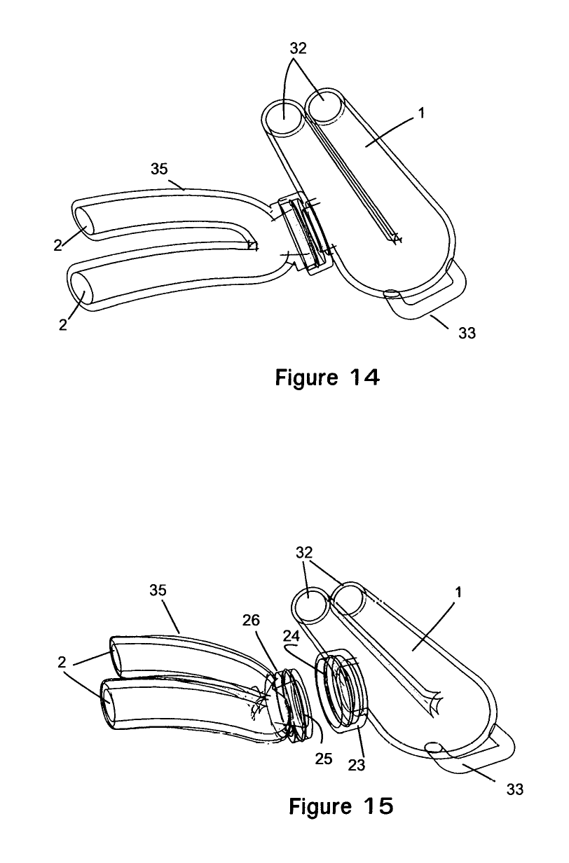

FIG. 14 is a bottom perspective view of the cannula used with the invention.

FIG. 15 is a perspective view of the cannula used with the invention showing the tube separated from the prongs. This view clearly shows how cannula tube 1 and collar ring 23 with “V” groove 24 snap onto inner ring 25 using the “V” ridge 26 of the nasal prongs 2.

As noted above, this allows for nasal prongs 2 to remain in place on patient while the cannula tube 1 can easily rotate 360 degrees in either direction to allow both hoses 3 to be on one side of the infant's head. This view also shows the soft polyurethane foam coating 35 on the nasal prong tips 2. When gently rolled between fingers and inserted into the infant's nostrils, the foam gently expands to fill the nostril allowing for a snug, air-tight fit.

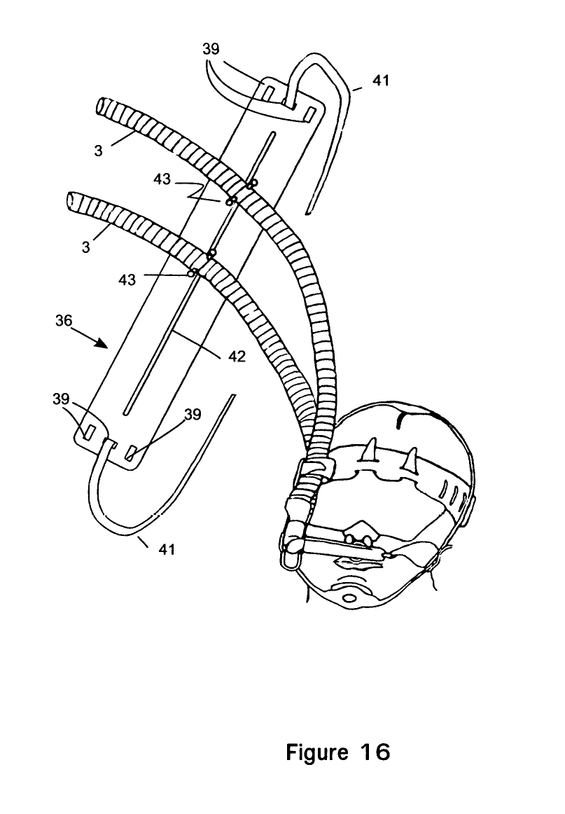

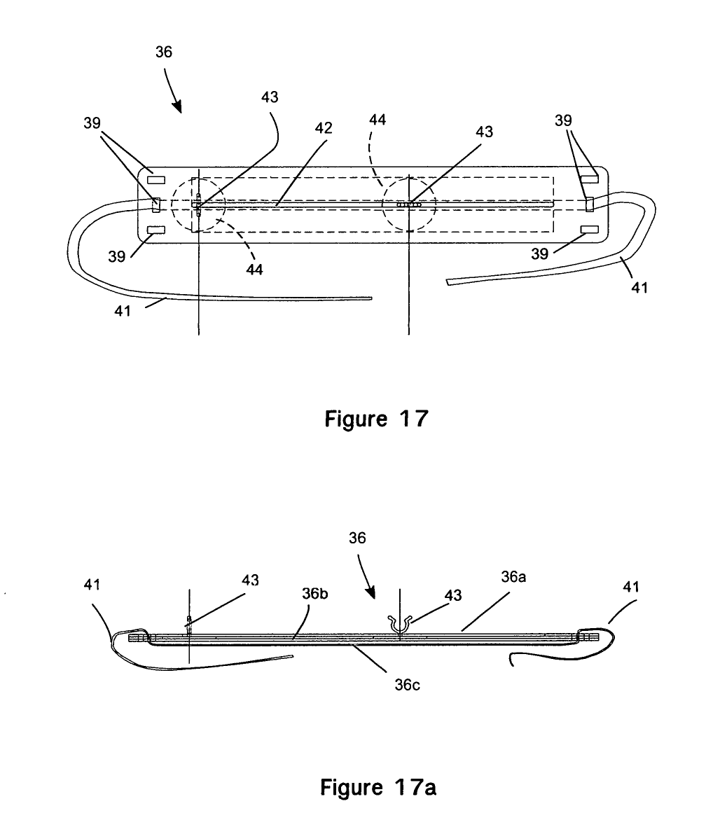

FIG. 16 is a perspective view of the embodiment of the invention of the CPAP cannula circuit stabilizer strap being used with the whole system. As discussed below, the strap 36 is comprised of a strap body that has three thin plastic strips 36a, 36b and 36c that are laminated together. In the preferred embodiment, the strips of plastic are approximately 12 inches long by 2½ inches wide. The laminated strips have a central area that is not laminated. This area has a slot 42 that allows at least one, and preferably two, “C” clamp(s) 43 to protrude through top plate 36a. Within the area are disks 44, which attach to the “C” clamps 43 (see e. g., FIGS. 17 and 22. The disks allow the “C” clamps to rotate and slide within slot 42 as needed. On each end of the strap 36 are a set of 3 holes 39, which allow for the insertion of a long hook and loop type fastener strap 41. The strap 41 is used to attach the strap 36 a bed or chair as suggested in the figure. The strap 41 and the holes 39 make up a means for securing the strap body to a fixture, such as a chair or bed part.

Note that the hoses 3 are secured to the “C” clamps 43 so that they are safely secured.

FIG. 17 is a top view of the stabilizing strap. This view shows the strap 36 and the top plate 36a, with the slot 42 formed therein. The disks 44 are shown in dashed lines below the slot 42 in the open space 45. This view also shows the three holes 39 and the strap 41, as discussed above.

FIG. 17a is a side view of the stabilizing strap 36. This view shows the three layers, 26a, 36b and 36c, all laminated together. It also shows the “c” clamps 43 extending outwardly from the strap 36.

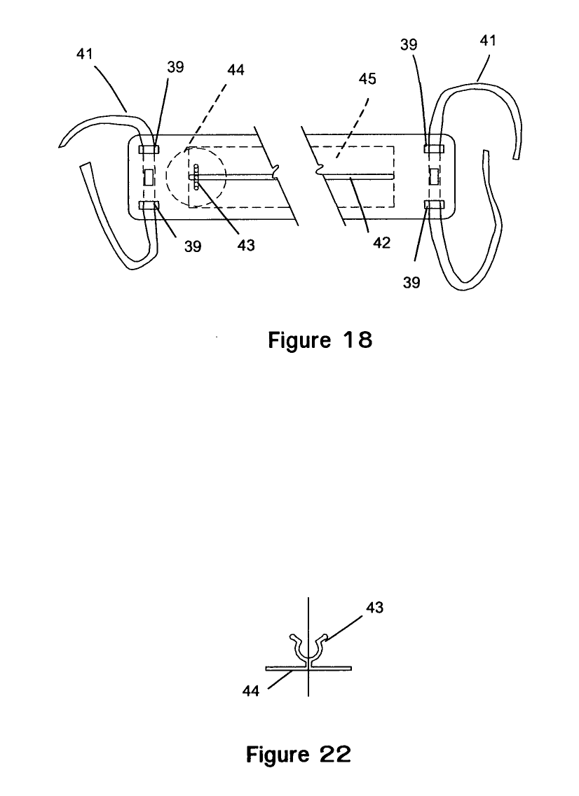

FIG. 18 is a top detail view of the stabilizing strap showing an alternative attachment structure. In this view, the strap 41 is threaded through the holes 39 (one on each end). The straps 41 can then be wrapped around a chair or bed part and stuck using the hook and loop type fastener that is on the straps 41 to secure the strap for use.

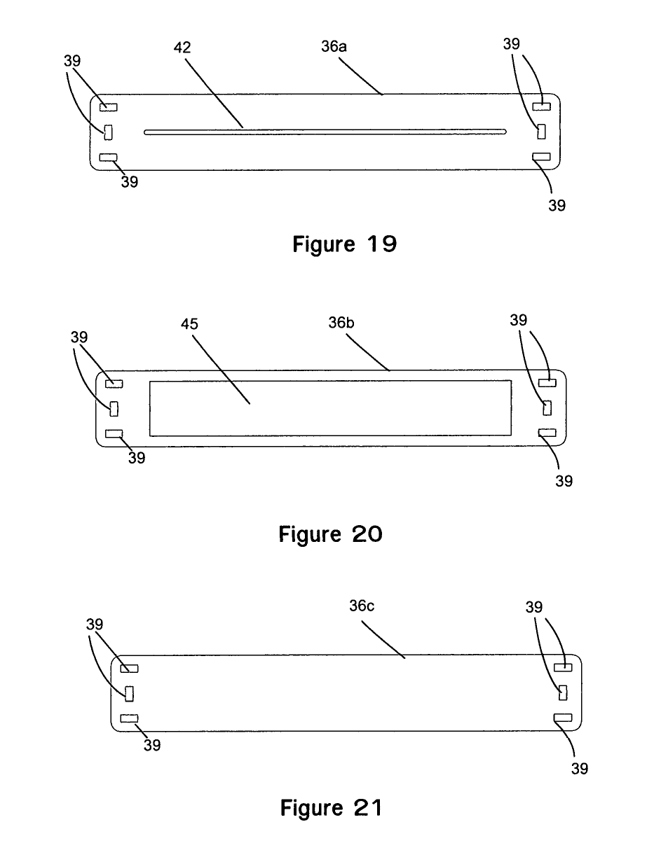

FIG. 19 is a top view of the top plate 36a used with the stabilizing strap 36. The top plate has a long slit 42 in the center allowing for “C” clamp 43 to slide freely therein.

FIG. 20 is a detail view of the center plate 36b used with the stabilizing strap 36. The center plate has a long wide cutout 45 in the center, which forms the open space when the plates are laminated together, that allows the round disk(s) 44 to slide and rotate freely when the three plates are laminated together. The “C” clamps are attached to the disks 44 for use.

FIG. 21 is a detail view of the bottom plate 36c used with the stabilizing strap 36. The bottom plate has the mounting holes 39 on each end.

FIG. 22 is a detail side view of a “C” clamp 43 and disk 44 used with the stabilizing strap 36. As noted above, the clamp 43 snaps around a hose 3. The disk 44 rests on top of bottom plate 36c and when the three plates 36a, 36b and 36c are sandwiched together, it creates a movable, but snug, fit for the “C” clamp 43 to move and rotate in any direction as needed.

The present disclosure should not be construed in any limited sense other than that limited by the scope of the claims having regard to the teachings herein and the prior art being apparent with the preferred form of the invention disclosed herein and which reveals details of structure of a preferred form necessary for a better understanding of the invention and may be subject to change by skilled persons within the scope of the invention without departing from the concept thereof.

I claim:

1. A cannula circuitry support system for use with continuous positive airway pressure machines used with infants in neonatal intensive care units utilizing at least one air supply hose comprising:
a) a hat with at least two vertically oriented slots formed therein, and at least two horizontal slots formed therein;
b) at least one elastic strap, threaded through said at least two slots in said hat, to secure said air supply hoses to the infant's head; and
c) a cannula, attached to said air supply hoses and having a pair of nasal prongs attached thereto to supply air to an infant patient.

2. The cannula circuitry support system of claim 1 wherein said hat further comprises: a pair of horizontal slots, positioned on said hat to receive an eye shield.

3. The cannula circuitry support system of claim 2 further comprising an eye shield having a soft, double-layered dark cotton fabric form and a pair of long, tapered strips, that slide into the pair of horizontal slots formed in said cap.

4. The cannula circuitry support system of claim 3 wherein the eye shield further comprises: a pair of plastic forms in the general shape of said long, tapered strips, sewn inside said fabric from at said tapered strips

5. The cannula circuitry support system of claim 1 further comprising: a chin strap, attached to said cannula to hold a user's mouth closed to prevent air leakage while the cannula is in use.

6. The cannula circuitry support system of claim 5 wherein the chin strap further comprises:
a) a soft double sided foam-lined cotton fabric material having two ends, each of said two ends having a slit formed therein;
b) a crown strap, having a center portion, and two straps extending outwardly therefrom, and further wherein each of two straps being sized to engage the slits formed in said chin strap; and
c) a hook and loop type fastener button, attached to said chin strap, said hook and loop type fastener button being in operative communication with an opposing hook and loop type fastener button attached to at least one layer of a hydrocolloid dressing, which adheres to the user's chin.

7. The cannula circuitry support system of claim 1 where said hat comprises:
a) a soft knit cotton hat having a top and a bottom edge and having a first pair of horizontal slots and a second pair of horizontal slots positioned above said first pair of horizontal slots, said first and second pairs of horizontal slots being centrally positioned on said hat to allow for attachment of an eye shield;
b) a first set of three vertical slots formed adjacent to said first and second pair of horizontal slots;
c) a second set of three vertical slots formed adjacent to said first and second pair of horizontal slots and being oppositely disposed from said first set of three vertical slots;
d) a third set of three vertical slots formed adjacent to a third and fourth pair of horizontal slots;
e) a fourth set of three vertical slots formed adjacent to said third and fourth pair of horizontal slots and being oppositely disposed from said first set of three vertical slots;
f) such that said thirds and fourth sets of three vertical slots and said third and fourth pair of horizontal slots are positioned above, and spaced apart from, said first and second sets of three vertical slots and said first and second pair of horizontal slots;
g) such that the bottom edge of said hat can be rolled up to allow the hat to fit a smaller user's head.

8. The cannula circuitry support system of claim 7 wherein the hat further comprises: a larger slot hole in the top of said had to allow a doctor to perform a palpable fontanelle check on said user without having to unstrap the hoses and remove said hat.

9. The cannula circuitry support system of claim 1 wherein said cannula comprises:
a) a tube having an attachment mechanism for receiving said at least one air supply hose, and a threaded portion formed thereon on and a rotating member attached to said threaded portion;
b) a pair of nasal prongs having a threaded portion formed thereon on and a rotating member attached to said threaded portion, such that said threaded portion on said tube is threadably engaged with the threaded portion on said second tube, and further wherein when said tube is attached to said pair of nasal prongs, said pair of nasal prongs is rotatable about an axis running through said threaded portions.

10. The cannula circuitry support system of claim wherein said pair of nasal prongs form two air channels extending upwardly from said threaded portion.

11. The cannula circuitry support system of claim 10 wherein said two air channels are coated with a layer of soft, hypoallergenic, open cell, polyurethane foam.

12. The cannula circuitry support system of claim 1 further comprising: a stabilizer strap to support said hoses apart from said cannula circuitry support system, said stabilizer strap including:
i) a strap body, having a slot formed therein;
ii) at least one “C” clamp for holding at least one air supply hose, slidably installed in said slot; and
iii) a means for securing said strap body to a fixture.

13. The cannula circuitry support system of claim 12 wherein the strap body comprises three thin plastic strips, a top strip, having a slot formed therein, a middle strip, having a cutout center portion, and a bottom strip, that are laminated together.

14. The cannula circuitry support system of claim 13 wherein said at least one “C” clamp is attached to a disk such that said at least one “C” clamp extends upwardly therefrom; and further wherein said disk is placed in the cutout center portion of said middle strip, such that said “C” clamp extends upwards through said slot in said top strip.

14. The cannula circuitry support system of claim 12 wherein the means for securing said strap body to a fixture comprise at least two holes formed in said top, middle and bottom strips, and a fastener strap that passes through said at least two holes.

15. The cannula circuitry support system of claim 12 wherein the fixture is selected from the group of a bed and a chair.
